(19) United States
(12) Patent Application Publication
(10) Pub. No.: US 2024/0112763 A1
(43) Pub. Date: Apr. 4, 2024

(54) SYSTEM AND METHOD ADAPTED FOR THE DYNAMIC PREDICTION OF NADES FORMATIONS

(71) Applicant: Clemson University, Clemson, SC (US)

(72) Inventors: Carlos D. Garcia Perez, Clemson, SC (US); Lucas de Brito Ayres, Clemson, SC (US)

(73) Assignee: Clemson University, Clemson, SC (US)

(21) Appl. No.: 18/473,255

(22) Filed: Sep. 24, 2023

Related U.S. Application Data
(60) Provisional application No. 63/409,549, filed on Sep. 23, 2022.

(51) Int. Cl. G16C 20/30; G16C 20/70

(52) U.S. Cl. CPC G16C 20/30; G16C 20/70

(57) ABSTRACT

A computerized system for generating digital representations of natural deep eutectic solvents comprising: a training dataset having a plurality of natural deep eutectic solvents; a set of non-stable natural deep eutectic solvents generated by random variation of the number of components, random variation of the individual chemical component, random variation of the stoichiometric coefficient for each component and any combination thereof; a set of non-transitory computer readable instructions, that when executed by a process are adapted to: receive a training dataset having a set of compounds using the simplified molecular-input line-entry system and having a designation of stable (e.g., 1) or not stable (e.g., 0), pre-training a language model according to the training dataset, fine tuning the language model according to a subset of labeled DES data, applying a classifier, and, providing results in a textual format.

### RELATED APPLICATIONS

This patent is a United States non-provisional patent application claiming priority from U.S. provisional patent application Ser. No. 63/409,549 filed Sep. 23,2022 titled System Adapted For The Prediction Of NADES Formations incorporated by reference herein.

### BACKGROUND OF THE INVENTION

### 1) Field of the Invention

A method and system for predicting the stability and other properties of previously unseen deep eutectic solvent mixtures for multiple uses to reduce the need for experimental (e.g., bench development) of mixtures and compounds.

### 2) Description of the Related Art

Historically, there has been interest in the development of eutectic mixtures such as ionic liquids (IL), deep eutectic solvents (DES) as well as natural deep eutectic solvents (NADES). These mixtures can be more environmentally friendly when compared to traditional organic solvents. These mixtures and alternatives can be applicable in a wide variety of industries targeting sustainable chemistry.

Some of the materials can be composed of specific ratios of two or three components and more, that can be in solid state and that lead to a material featuring a melting point that can be significantly lower than the melting points of the individual components. One desirable property is that the mixture forms a stable liquid at room temperature. It is also desirable to have a careful selection of the physico-chemical characteristics of the components (molecular weight, hydrogen bonding, pKa, etc.) that can enable the formation of solvents having different properties (e.g., stability, viscosity, polarity, and density). Properties that can provide advantages with customizing solvents towards specific applications including biocatalysis, chromatography, extraction media, electrochemistry, as well as pharmaceutical ingredients to enhance therapeutic properties are desirable. In some opinions, solvents that are DES can be more environmentally friendly than IL due to their intrinsic properties such as biodegradability, low or non-toxicity, easy preparation with no purification steps, as well as inexpensive starting materials. Further, NADES can be formed using natural compounds commonly present in biological systems, including plants.

One of the disadvantages with DES and NADES is that their development is typically empirically driven. Because there are only general guidelines to predict their formation, new DES are often derived from structurally similar components rather than being drastically new solvents. Examples of these include families of DES based on choline chloride and similar carbohydrates or similar organic acids. A problem in the industry and with the current technology is that bench-top trials of new mixtures are time-consuming, labor-intensive, and expensive even at a lab scale and therefore limit the development of DES.

It would be advantageous to have computational tools to provide insights into the relationship between chemical structure and properties of NADES/DES and, consequently, guide the application of these mixtures. One attempt to solve these issues is to use thermodynamic modeling such as Perturbed Chain-Statical Associating Fluid Theory (PC-SAFT) to Atomistic modeling methods that can include Density Functional Theory (DFT) at the quantum level. Unfortunately, while these models can explain the formation of some DES, they require specialized knowledge to build and are not yet able to make statistically validated predictions of new mixtures. There has also been some interest to use machine learning approaches based on artificial neural networks (ANN) are also frequently used as an auxiliary tool to predict properties of solvents. However, due to the complexity of these deep learning architectures, a substantial volume of data is required to train the model, to properly adjust the parameters of the neural network (e.g., weights and bias), and to extract meaningful information from the chemical space.

The advances of artificial intelligence (AI) can produce algorithms to perform tasks such as driving cars, playing complex games, composing classical music and even generating realistic images by using text as input parameter. Some of these abilities are linked to the implementation of deep neural network architectures in combination of the use of large databases as well as the increase in computing power. This strategy has also been showing promise in several sub fields of natural sciences such as chemistry, biology, and physics through speech recognition, data analysis, and computer vision. There have been some attempts to use deep learning to predict properties of molecules and to predict a number of chemical reactions, these advances have always been linked to large databases containing millions of entries associated to a single problem. It would be advantageous to have a system that can predict stability and/or other attributes to a resulting DES, including NADES, without the need of these large databases containing millions of entries. The ability to generate potential compounds in this manner is an area that has not received needed attention.

Traditionally, the need for large databases has limited the technology since these large (e.g., million or more record) databases are not always available and performing experiments to determine chemical compounds is an unreasonable task at the human time scale.

Therefore, there is a need for a system that can provide quick and efficient potential success compounds, especially those that are time consuming and with unpredictable results with minimal laboratory experimentation.

### BRIEF SUMMARY OF THE INVENTION

The above objectives are accomplished by providing a computerized system for generating digital representations of deep eutectic solvents comprising: a training dataset having a plurality of deep eutectic solvents; a set of non-stable deep eutectic solvents generated by random variation of the number of components, random variation of the individual chemical component, random variation of the stoichiometric coefficient for each component and any combination thereof; a set of non-transitory computer readable instructions that when executed by a process are adapted to: receive a training dataset having a set of compounds using the simplified molecular-input line-entry system and having a designation of stable (e.g., 1) or not stable (e.g., 0), pre-training a language model according to the training dataset, fine tuning the language model according to a subset of labeled NADES data, applying a classifier, and, providing results in a textual format.

The present system provides for the development of a transformer-based neural network model capable of predicting stability of potential eutectic mixtures at different stoichiometric ratios by means of simplified molecular-input line-entry system (SMILES) representations, rather than an extensive set of physicochemical parameters as input. This system allows the use of relatively small datasets, which also reduces training time, model complexity, and computational cost. This system is novel, at least because it uses transformers and a language-based approach towards the screening of new deep eutectic solvents. The system can include pre-training a transformer model by using unlabeled general chemical data and then, fine-tuning of the last layer of neurons in the model to perform a binary classification using labeled chemical data related to DES. The system demonstrates a satisfactory performance (accuracy and F1-score=0.82), allowing the prediction of multiple stable natural eutectic mixtures (n=200) from a general database. The validity of such predictions has been verified by comparing the stability those NADES against those reported in previous publications as well as by the development of a stable DES, containing ibuprofen.

### BRIEF DESCRIPTION OF THE SEVERAL VIEWS OF THE DRAWINGS

The construction designed to carry out the invention will hereinafter be described, together with other features thereof. The drawings represent computer readable instructions and methods and processes or operation that are included in the system. The invention will be more readily understood from a reading of the following specification and by reference to the accompanying drawings forming a part thereof, wherein an example of the invention is shown and wherein:

FIG. 1 is a schematic of aspects of the system.

FIG. 2 represents the learning process of the general chemistry model of the system.

FIGS. 3A and 3B are graphical representations of aspects of the feature and functions of the system directed to the implementation of the fine-tuning methodology.

FIGS. 4A and 4B are graphical representations of aspects of the feature and functions of the system directed to the implementation of the fine-tuning methodology.

FIG. 5 is a graphical representation of aspects of the features and functions of the system directed to the implementation of the fine-tuning methodology.

FIG. 6 is a schematic of aspects of the system.

FIG. 7 are examples of the application of the system.

FIG. 8 is a schematic of aspects of the system.

### DETAILED DESCRIPTION OF THE INVENTION

With reference to the drawings, the invention will now be described in more detail. This system can include a transformer-based neural network model capable of predicting stability of potential eutectic mixtures at different stoichiometric ratios by means of simplified molecular-input line entry system (SMILES) representations, rather than an extensive set of physicochemical parameters as input. This system allows the use of relatively small datasets, which also reduces training time, model complexity, and computational cost. The system is an automated method to predict the formulation (type of component and amount) needed to render NADES.

This system can provide for the use SMILES combined with computer readable instructions, including computer readable instructions for natural language processing (NLP) tools that can be used to transform a general computer system into a specialized computer system that can conduct digital chemical compound creation and analysis. Using the system herein, the computer readable instructions can be adapted to extract information from chemical subject matter databases and data and can use text representing compounds. Therefore, the system herein can be used to transform a computer system, train the system, and generate a chemistry model in a short period of time. In one embodiment and applications, the training time is less than 12 hours. In one embodiment, the system can achieve a learning rate (e.g., training loss <3.5) after the epoch (iteration) 20. Further, the system can include a layer of neurons of a resulted model that can be trained to use a relatively smaller database (e.g., containing less than 1000 entries in one embodiment) using learned information in a specific field of chemistry. The system includes a design to predict the stability of eutectic mixtures, using text representation. The system can include computer readable instructions and datasets that can be used for training a neural network model using general unlabeled chemical data from organic reactions. In sequence, the last layer of neurons from the model can be fine-tuned into a binary classifier using labeled NADES/DES dataset. Additionally, an auxiliary software (uACL) can be used to query the probability of any mixture to form a stable NADES (developed classifier) and then export the results (e.g., mixture composition, stoichiometric ratio, and probability of stability) in a variety of file formats include CSV.

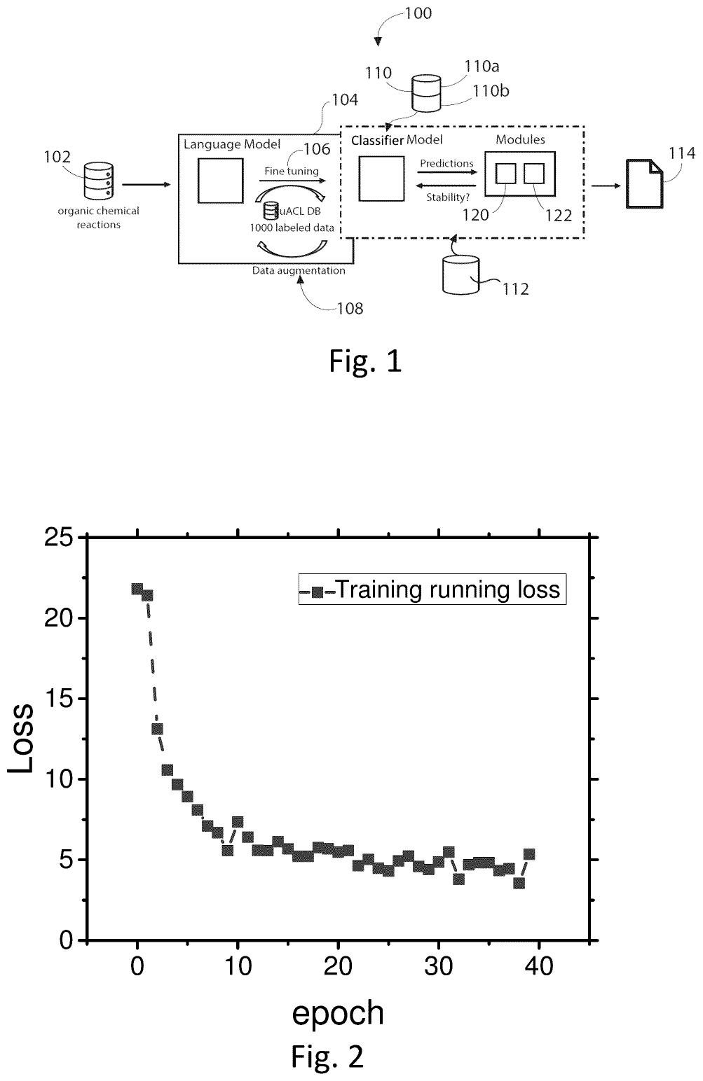

Referring to FIG. 1, system 100 is shown schematically and illustrates examples of the modules and the work sequence. In one example for illustrative purposes, the system used approximately one million unlabeled organic chemical reactions 102 such as the USPTO_MIT dataset represented in the SMILES canonical notation used as text dataset to pre-train the transformer-based neural network. This initial process can use a large amount of unlabeled data since the neural network seeks to model a language based on text sequences within specific contexts. The process can include adjusting the intrinsic parameters at 106 such as weights and bias of the data to reduce a loss function which can results in the pre-training of the model at 106 and matching the predicted results with the actual resulted results to improve the natural leaning and artificial neural network algorithms in the neural network. The process can include using computer readable instructions in a second database 110 to fine tune the neural network at 106. The neural network can then receive an initial database of existing deep eutectic solvents 106. The second database can be divided into a training portion 110a and a testing portion 110b.

Once the model has been provided, an initial group of molecules 112 can be received by the neural network and the model can calculate the probability of formation for a set of mixtures 114. The set of mixtures can include associated information such as their probability of formation for each artificial neural network algorithm. A mixture can be selected from the set of mixtures predicted by the set of artificial neural network algorithms wherein the first mixture comprises molecules not present in the training portion. The computerized method can compare a test mixture within the set of mixtures with the testing portion to provide a confidence score for each artificial neural network algorithm in the set of artificial neural network algorithms. The set of artificial neural network algorithms in confidence score order to a use so that one or more artificial neural network algorithms with the highest probability of producing deep eutectic solvents having certain attributes can be provided.

The loss versus epoch graph for the pre-training step is shown in FIG. 2. As shown, the loss function can dramatically decrease from epochs 0 to 10 and then remains relatively constant until the end of the training process. The average loss for the training dataset in one embodiment was 3.87, while the average loss for the evaluation dataset was 4.00, showing that the system performance of the neural network using both datasets reached a convergence point meaning that additional processes for training will not lead to an improvement in the general chemistry model. When reached, the training process can be stopped, and the generated model can be tuned under several augmented data conditions. The computerized method can include a mixture generator 120 and a compound finer 122.

In one example, a database contains over 10 previously reported examples of NADES/DES, where the components are represented using the canonical SMILES notation. Those combinations leading to stable mixtures (e.g., synthesized NADES and/or DES in the liquid state that are stable for more than one week at room temperature) were labelled as “1”. Combinations of components not leading to liquid mixtures, or those that crystalize soon after the synthesis (non-stable) were labelled as “0”. The entries into the database can be divided into training and fine-tuning. In one embodiment, 80% of the database was used to generate a training dataset and the remaining 20% was used as evaluation dataset, to fine-tune the general chemistry model into a binary classifier, capable of classifying mixtures as stable (1) or non-stable (0). Generally, the binary classifier is adapted to categorize the compounds into one of two distinct classes or categories (e.g., stable, or unstable). The binary classifier can provide decisions or predictions about which class a given input data point will belong to. For example, given a specific module input, the binary classifier can determine whether a resulting compound would be stable or unstable and provide a confidence value as to the outcome (e.g., XX% chance of being stable).

The fine-tuning step can be carried out by using a database containing approximately 1000 labeled mixtures of DES and NADES, in one example, taken from available sources. Within these, 800 mixtures are stable (labeled as 1), and 200 mixtures are not stable (labeled as 0). While this imbalance reflects what is normally published (mostly positive results), its weight on the dataset is undesirable and can lead to overfitting problems, impacting the performance of the classifier (e.g., overoptimistic estimation). To overcome this issue, the system can implement a data augmentation strategy by generating random NADES and DES mixtures in the training dataset and then labeling them as zero (unstable). For example, varying the stoichiometric coefficient between 1 and 10 for 198 compounds, can lead to 40 million possible combinations only for ternary mixtures

$$( \frac{618 !}{3 ! \times 615 !} ) .$$

In this sense, the effectiveness of this strategy was mainly assessed by evaluating two parameters: Matthew's correlation coefficient (MCC) and loss function. For both cases, the training dataset was augmented by adding synthetic data (1, 5, 10, 25, 50, 100, and 500) while the test dataset remained constant with 200 mixtures.

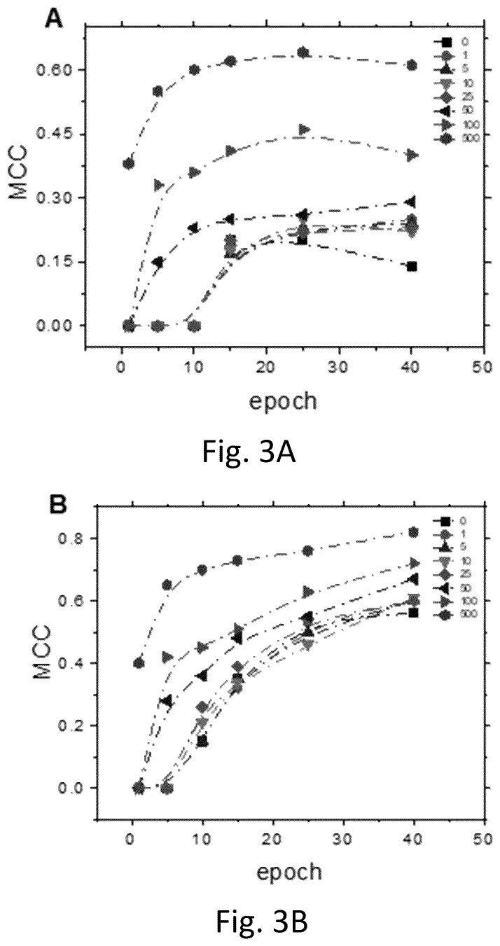

The MCC is applied to the system and results in a reliable metric for binary classification problems where the dataset available for training as well as fine tuning are unbalanced. This application takes into consideration all the categories in the confusion matrix (true positive, false negative, true negative, and false positive) to compute the correlation between the predicted value by the classifier with the true one. This correlation ranges from −1 to +1, where −1 indicates total disagreement, 0 indicates no correlation, and +1 indicates total agreement. The effect of several training data augmentation on the MCC metric versus number of epochs is shown in FIGS. 3A and 3B. The effect of training data augmentation (1, 5, 10, 25, 50, 100, and 500) on MCC versus number of epochs number for validation dataset (FIG. 3A) and training dataset (FIG. 3B) is shown. The MCC value represents the agreement between the predicted value and the true class, where: +0.01 to +0.19 indicates no or negligible relationship, +0.20 to +0.29 indicates weak positive relationship, +0.30 to +0.39 indicates moderate positive relationship, +0.40 to +0.69 indicates strong positive relationship, and +0.70 or higher indicates very strong positive relationship.

For the testing dataset (FIG. 3A), the MCC rises as the epoch number increases, reaching a plateau in iteration number 15 for all augmented data scenarios. Additionally, it is interesting to note that the MCC is improved as the training synthetic data is incremented, accomplishing a satisfactory performance (MCC higher than 0.40) by using 100 and 500 augmented data after the iteration number 15. On the other hand, the model's performance evaluating the training dataset (FIG. 3B) is already satisfactory even by not implementing any synthetic data. This is expected since the same dataset was already seen by the deep neural network during the fine-tuning process.

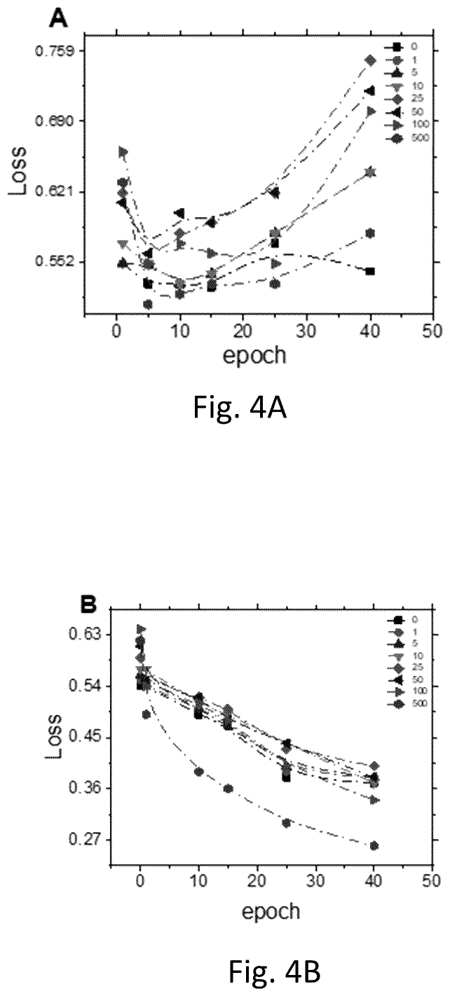

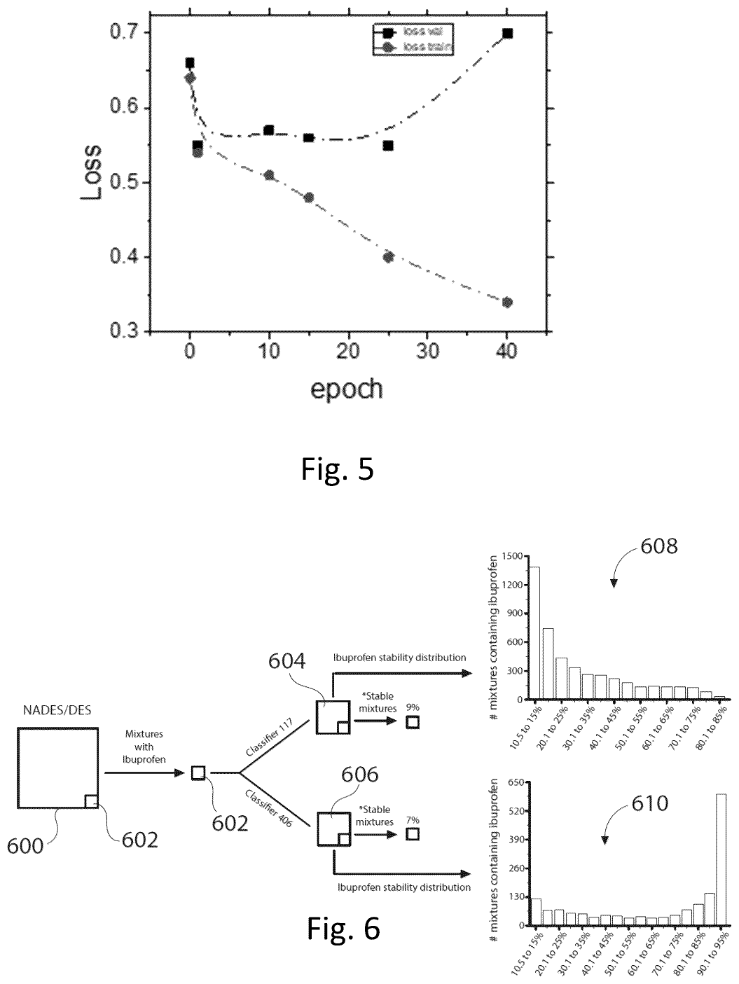

The system measurement of the loss was performed aiming to elucidate the effect of the implemented data augmentation strategy on the classifier's performance in terms of underfitting as well as overfitting. The results are shown in FIGS. 4A and 4B which illustrate loss versus epoch graph for validation dataset (FIG. 4A) versus training dataset (FIG. 4B). The system therefore provides a classifier that starts to overfit after the iteration number 15 regardless of the amount of data augmentation (FIG. 4A). On the other hand, this issue is not present upon assessment of the training dataset (FIG. 4B) where the loss decreases as the number of synthetic data is added. The overfitting issues are evidenced when the loss for the evaluation and training dataset is plotted together for the same number of augmented data as shown in FIG. 5 illustrating loss versus epoch number for both evaluation (top line) and training dataset (bottom line).

As shown from the system results, both losses were at the same point (approximately 0.67) and then started to diverge as the epoch number increases, reaching the maximum difference at epoch forty. At this point, the model is overfitted and generalizes unseen data poorly. On the other hand, the classifier trained only with 5 epochs, for example, is underfitted. In this case, the training time is too short, and the model is not able to get meaningful information from the chemical space. In this context, an optimum classifier can be shown to be trained with the number of epochs in between these two extremes, where the number of interactions is enough to learn important information from the dataset but not too long to learn noise as well as not useful information.

After the assessment of MCC and loss described above, a classifier 117 (also referred to as classifier alpha) was included in the system to be the implemented model. This classifier was fine-tuned by using the training data set augmented with 100 synthetic data while the number of epochs was fixed at 15. For comparison purposes, another classifier (classifier 406, also referred to as classifier gamma) was also fine-tuned by using the same augmented dataset but with the number of epochs set up to 40. Both classifiers were used to predict the stability of 1 million non-labeled eutectic mixtures (e.g., NADES/DES universe) randomly generated by the uACL computer readable instructions. It is advantageous that mixtures present in the NADES/DES universe be generated completely randomly rather than fixing it is constituents to a specific component such as ibuprofen or any other chemical. Posterior to the predictions, only the results (mixture in the smiles representation, probability of being stable, probability of not being stable, and label) for mixtures containing ibuprofen were post processed and then exported.

Referring to FIG. 6, the computerized method for predicting mixtures with ibuprofen. A set of potential mixtures 600 that could include ibuprofen is provided. A subset of mixtures 602 is identified and a first artificial neural network algorithm 604 is used to predict stability for a set mixture and a second artificial neural network algorithm 606 used to predict stability for the same set of mixtures. The results are shown respectfully as 608 and 610. The probability distribution of stability for mixtures containing ibuprofen predicted by both artificial neural network algorithm (e.g., classifiers) predicted by the classifier 604 and by the classifier 606. In this example, the system results in approximately 2% out of the NADES/DES universe presented ibuprofen on its composition. Within these, 9% of those mixtures were predicted to be stable by the classifier 604 while this percentage dropped to 7% by using the classifier 606. Note that this example focused the analysis on mixtures featuring the probability of being stable higher than 50.1%. Therefore, the number of stable solvents could considerably decrease which can result in an increased relative chance of finding one or more stable mixtures, if this cut-off was set to 70%, for example. Additionally, the database used for generating those random mixtures is biased with classical compounds (e.g., Hydrogen bond donor and acceptor) to produce eutectic solvents. As a point of reference, the same strategy was implemented by using an open-source database of natural compounds 70 and the stability found to be less than 1%.

Classifier 604 was also shown to have a decreasing stability distribution, presenting only 24 mixtures at a range of stability between 80.1% and 85%. On the other hand, the classifier 606 has an increasing stability distribution, presenting more than 600 stable mixtures at a range of stability between 90.1% to 95%. This occurs since this classifier was trained to be overfitted although it's MCC has been the same as the classifier 604 (0.42) because the number of training iterations plays an important role during the development of an optimal classifier. Moreover, from a statistical point of view, it is more likely that the number of eutectic mixtures decreases as the probability of being stable increases as exhibited by the classifier 604. The top 10 stable mixtures containing ibuprofen predicted by this classifier is shown in Table 1.

TABLE 1

| | | | | | Probability |
|---|---|---|---|---|---|
| MIXTURE | Component | Component | Component | Molar | of being |
| # | 1 | 2 | 3 | ratio | stable |
| 1 | 2-diethylaminoethanol | Ibuprofen | Glycol | 1:2:1 | 84.48% |
| 2 | 1,6-Hexanediol | Ibuprofen | Methanol | 1:1:3 | 83.85% |
| 3 | Thiocyanic acid | Zinc | Ibuprofen | 1:2:4 | 83.61% |
| | | Chloride | | | |
| 4 | Methanol | Undecanoic | Ibuprofen | 2:1:1 | 83.41% |
| | | Acid | | | |
| 5 | Proline | Zinc | Ibuprofen | 2:2:1 | 83.33% |
| | | Chloride | | | |
| 6 | Undecanoic acid | Ibuprofen | Glycol | 1:1:5 | 82.52% |
| 7 | Proline | Ibuprofen | Diethanolamine | 1:3:3 | 82.28% |
| 8 | Sodium acetate | Methanol | Ibuprofen | 1:2:1 | 82.00% |
| 9 | Choline Chloride | Ibuprofen | Glycol | 1:3:4 | 81.23% |
| 10 | Hexitol | Choline | Ibuprofen | 2:1:3 | 80.84% |
| | | Chloride | | | |

The solvents presented in Table 1 are ternary mixtures with well-known hydrogen bond acceptors as well as hydrogen bond donors on it is a composition such as chloride derivates, alcohols, acids, and polyethylene glycol. In contrast, most of the unstable solvents precited by this classifier are quaternary and/or quinary mixtures with a high number of molar ratios.

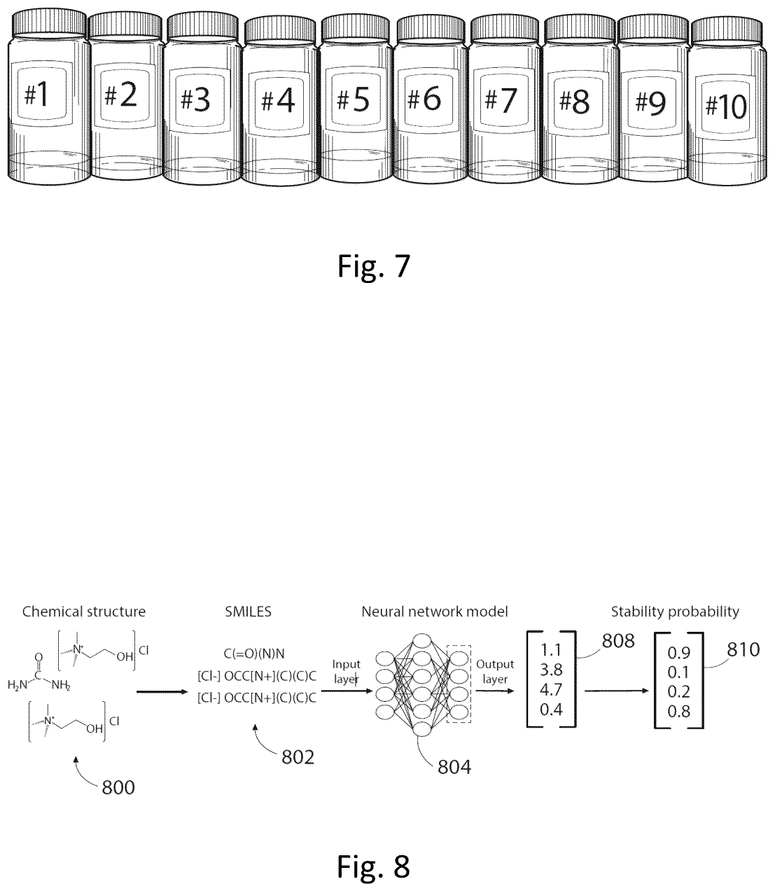

In one example, for analyzing the system, combination number 8 was selected to be experimentally assessed as a first example of the proposed strategy to predict sable eutectic solvents. This mixture is composed of Ibuprofen, sodium acetate, and methanol at molar ratio 1:1:2 respectively. The synthesized solvent and the chemical structure of their constituents is shown in FIG. 7 showing eutectic mixture (A) composed of ibuprofen (B) sodium acetate (C), and methanol (D) at the stable molar ratio 1:1:2. These images represent experimental validation of the formation of 10 NADES predicted by classifier 604. The composition of each mixture is described in Table 1.

In one example, the experimental results produced eight out of the ten mixtures (80%) being stable NADES, remaining in clear liquid form at room temperature for at least one week. This result correlates to the average predicted stability score of these mixtures (82.8%) by the systems and provides some indication that the predicted stability scores may be a good proxy for the actual probability of forming a stable NADES. The system resulted in neither under-nor overfitting and with a relatively unbiased training dataset—a classifier's prediction scores should be a good approximation of true probability. The system can also provide a more extensive list including all 24 mixtures or more at a range of probability for a stable NADES between 80.1% and 85% could provide additional stable formulations.

Furthermore, when one of the classifier's performances was tested on the validation dataset, its overall accuracy was calculated at about 82% showing that the performance on the held-back evaluation data corresponds very well to the model's true predictive power. In one example to verify the system, 10 combinations most likely to form stable NADES (Table 1) were prepared in the laboratory. In cases, the corresponding amount of the pure constituents were mixed in a sealed glass vial and incubated at 80° C. (in a water bath), under gentle stirring, for approximately two hours. This process rendered liquid mixtures that were then removed from the water bath and placed on the bench, where they were kept at room temperature for (at least) a week. It is also important to note that those mixtures formed with methanol (marked as * in Table 1) are not strictly considered natural and would not be applicable towards pharmaceutical preparations. Nevertheless, these mixtures were evaluated and used to validate the predictions of the system. One of the resulting eutectic mixtures is a clear liquid with low viscosity and does not present crystallization at room temperature for over one week. The melting point of sodium acetate is 324° C., which suggests that the combination of ibuprofen and methanol hinders the crystal lattice formation by donating hydrogen bond. Moreover, a eutectic mixture with the same molar ratio of ibuprofen and sodium acetate was synthetized in absence of methanol and proved to be stable although it presents higher viscosity. In this sense, the use of methanol corroborates the fact that tertiary components can be used to adjust the viscosity of NADES as well as DES.

The performance of the classifier 604 was also investigated by means of the evaluation dataset use. This dataset is composed of over 145 mixtures containing stable and unstable eutectic mixtures. The results are shown in Table 2.

TABLE 2

| | | | F1- | |
|---|---|---|---|---|
| Classifier | MCC | Accuracy | score | loss |
| 604 (Optimum) | 0.42 | 0.82 | 0.82 | 0.56 |
| 606 (Overfitted) | 0.42 | 0.73 | 0.73 | 0.70 |

Taking into consideration all the parameters described above the classifier 604 presented a satisfactory performance evaluating a dataset never seen before by the model. This performance could be improved by increasing the quality as well as the amount of data present in the training database. On the other hand, the training time would increase, and this strategy would be less attractive even for high-end personal computers.

The predictive power of this system can be modified with the accumulation of more data, and the system itself can be used to optimize the process. Through the process of bench-testing the model's predictions and thereby increasing the amount of available training data, the model can improve accuracy through subsequent rounds of training. Techniques to optimize this process, known as Active Learning, typically rely on bench testing either the least-confident predictions (i.e., those predictions that lie very close to the chosen 0.5 threshold for stability), on a maximally diverse group of test cases, or a combination of these. The result of this process, if good balance is maintained in the bias of the dataset and care is taken to avoid under- or overfitting, could be the overall accuracy of prediction as well as the confidence score in individual predictions rising from around 82% to 90% or higher. The confidence score can be determined from the fit between the predicted compounds and the testing portion of the dataset.

An illustration of the system is shown in FIG. 8. In one embodiment, the results of the system can be generated using a high-performance computing community and cluster, such as the Clemson University Palmetto Cluster. In one embodiment, a NVIDIA Tesla V100 was used as graphical processing unit (GPU) to train and fine-tune the deep learning model. In one embodiment, the ELECTRA deep learning transformer from Hugging Face was used to train a general chemistry model as well as to fine-tune the model to enable performing downstream tasks such as binary classification. The system can include pretraining a discriminator transformer model that predicts tokens that are determined to be replaced or not from another neural network called generator. This system allows the development of small models that still perform well compared to traditional state-of-the-art natural language processing models such as GPT, BERT-Base, and RoBERTa, given the same dataset. This unique feature of the system allows the use of relatively small datasets as well as less computational power to train accurate models.

In one embodiment, the USPTO_MIT database was included to train the general chemistry model. The database of this embodiment included approximately a million organic reactions, represented by the SMILES notation. Each line of the source database that contains reactants can be linked to its corresponding products on the target database. These two data sets (e.g., text files) can be merged into a single raw database where the reactants are separated from the products by a non-SMILES character “>” 80% of this database was assigned as the training dataset and the remaining 20% was used as evaluation or testing dataset, for the proposed general chemistry model.

In one test example, a uACL database can contain approximately 1000 previously reported examples of NADES/DES, where the components are represented using the canonical SMILES notation. Those combinations leading to stable mixtures (e.g., synthetized NADES and/or DES in the liquid state that are stable for more than one week at room temperature) were labelled as “1”. Combinations of components not leading to liquid mixtures, or those that crystalize right after the synthesis (non-stable) were labelled as “0”. Again, 80% of this database was used to generate a train dataset and the remaining 20% was used as evaluation or testing dataset, to fine-tune the general chemistry model into a binary classifier, capable of classifying mixtures as stable (1) or non-stable (0).

When comparing the system with experimental results, when the system has a limited size of the database, (e.g., containing ˜1000 examples of previously reported NADES, the system led to significant overfitting. The system can obtain relatively high scores, even if predicting “stable” for non-stable mixtures. To respond to this occurrence, the uACL database can be augmented by the script “mixture generator alfa”. The script is responsible for generating mixtures by randomly varying its size in terms of number of components (e.g., from 3 to 5), varying the individual chemical component itself (providing 198 possibilities in one example) as well as its stoichiometric number (e.g., from 1 to 10). The mixtures generated by the system in the example were labeled as “0” (unstable) and then added to the uACL database according to the number of data augmented (e.g., . . . 25 100, 500).

A chemical structure 800 can be converted to the SMILES format at 802. This information can be provided to a neural network 804 having one or more algorithms. The neural network can provide a mathematical function 808 that can be converted into vectors of probabilities 810. The probabilities can represent the probability of stability for a given mixture. In one embodiment, the computer readable instructions includes several hidden layers for the generator as well as discriminator for the ELECTRA deep learning model were 4 and 16, respectively. In this example, the vocabulary size is set to 30,000 arguments and the number of training epochs adjusted to 40. The resulting textual file was used as a train dataset while an evaluation file was used as an evaluation dataset. The system can contain all the trained parameters (e.g., discriminator, generator, vocabulary, and pytorch model) and can be stored in a single directory.

Computer readable instructions to fine-tune a language model of the system into a binary classifier can be created. One layer of neurons of the model can be fine tuned into a binary classifier by using the training database as training dataset and the evaluation data as an evaluation dataset. Additionally, the augmented train datasets that are generated can be used to investigate the performance of those models given the same evaluation dataset.

To predict the stability of unseen mixtures by the classifier model, the system can include three main modules: mixture generator beta, classifier model, and compound finder. The mixture generator is responsible for generating random mixtures with random number of components, random components as well as random stoichiometric number. Differently from the mixture generator alpha described herein, the beta version will not assign any label to the combination generated and all the results can be saved in a text file. This text file can be sent to the classifier model that will infer the probability for each mixture to either stable or not. In one embodiment, this was accomplished by implementing a SoftMax function on the raw output of the last layer from the deep neural network model. Predictions with respective stability scores are postprocessing on the compound finder module. This module allows the user to export results (e.g., mixture, stability, and label) exclusively to a single compound (e.g., only mixtures that contains Ibuprofen in its combination) in a variety of formats including the CSV format.

### EXAMPLE 1

In development, testing and operating the system the following example is illustrative of the system. Solid ibuprofen can be purchased from several suppliers that can include Spectrum Chemical Mfg. Corp. (New Brunswick, NJ, USA). Sodium acetate can be purchased from several suppliers including Sigma-Aldrich (Burlington, WI, USA) and methanol can be purchased from several suppliers including Thermo Fischer Scientific (Fischer Chemical, NJ, USA). These reagents can be analytical grade or better. Prior to the preparation of NADES/DES mixtures, all the individual solid samples were heated at 40° C. overnight for removal of any water residue. NADES and/or DES with molar ratio compositions predicted by the artificial neural network model were prepared by the traditional heating method (85° C.) under magnetic stirring (350 RPM) for 2 hours and then allowed to cool down to room temperature.

The present system provides for computationally and energy efficient approach for formulating new natural deep eutectic solvents (NADES). In some industries, forming these solvents would be the first step towards their application in the pharmaceutical, agricultural, industrial, and or food industries, to name a few. Towards that goal, a transformer-based neural network model can be pre-trained to recognize chemical reaction patterns from SMILES representations (unlabeled general chemical data) and then fine-tuned to recognize the labelled patterns of mixtures known to lead to the formation of either stable or unstable eutectic solvents using binary classification. This strategy, using a comparatively small database (e.g., 1000 inputs) and a data augmentation strategy, enabled the prediction of multiple new stable eutectic mixtures (e.g., n=337) from a general database of natural compounds.

An example was used for validating the training process as well as the results of the prediction (components and molar ratios) needed to render NADES with ibuprofen, a molecule that was not present in the original database. Examining the results, in one test example, the 10 mixtures with the highest predicted likelihood of forming stable NADES were prepared, rendering a success rate of 80%; a figure which strongly validates both the overall accuracy of the model (calculated at 82% on the validation dataset) and the model's confidence that individual mixtures will be stable (a predicted mean of 82.8% for the tested mixtures). The system can provide liquid preparations of ibuprofen and other bioactive compounds that can significantly impact the pharmaceutical and nutraceutical industries, as the absorption of many drugs and natural bioactive compounds have been historically hindered by solubility issues. More importantly, this system can provide transformative solutions to the pharmaceutical and nutraceutical industries, where bioactive compounds can become functional components of liquid formulations, rather than simple solutes dispersed in a NADES matrix. This system represents a leap forward towards the efficient development of the newest class of DES, therapeutic DES, or THEDES.

### EXAMPLE 2

The system can be used for the prediction of chemical reactivity, as applied to control lipid oxidation such as would be advantageous in the rendering and pet food industries. The system can provide for specific mixtures of molecules having synergistic interactions that can enable lowering the dose used (minimizing taste interferences and customer concerns) as well as significantly extending the lifetime of these products. The dataset included in the system can be used for determining previously unknown molecule candidates and synergistic combinations that can be used, for example, in the rendering and pet food industry. The system can determine and propose previously unknown molecule strategies for the preservation of rendered fats and meals that can be utilized in pet and livestock feed, thus providing the basis to rationally lower the concentrations used and to either supplement or replace current molecule formulations. The system can determine and provide potential interactions, such as hydrogen bonding and van der Waals interactions, by us of a general foundational chemistry model and adopted it into a regressor with the ability to predict the behavior (e.g., antagonistic, additive, or synergistic) of molecule mixtures.

The system and process can include a foundational chemistry model 600 that represents basic chemical reactions and compounds derived from an initial training set of data 602. The database can then be modified to include a set of neural network algorithms such as regressors 604. Each regressor can include computer readable instructions that provide the statistical probability of a given mixture (e.g., input) to have antagonistic, additive, synergistic behavior, or other attributes of a compound (e.g., output). Once generated, each regressor can be compared to a corresponding dataset representing non-generated compounds including a comparison of the predicted attributes and behavior against the known attributes and behavior. The comparison and its results can be measured using metrics such as root-mean-square error (RMSE), mean absolute percentage error (MAPE), as well as R². The results can be used to rank the regressors and select the preferred regressor according to the purpose. For example, if the desire is to find an advantageous combination index (CI), one or more regressors that produce a most closely matching compound that is known, not generated by the regression, from the initial molecule, can be selected. Therefore, the system can be adapted to provide regressors which provide the closest predictions of synergistic, additive, antagonistic effects, or other attributes from combining two or more drugs or compounds can be used for prediction of additional compounds using the selected regressors. In one embodiment, the regressors predictive CI compared to actual CI is shown in FIGS. 9A (predictive CI) and 9B (actual CI). The one or more select regressors, according to the comparison of the predictive CI with actual CI, are then used in the system for the predictions of the CI values of various mixtures of phenolic molecules.

In one embodiment, the system can include a process that departs from the natural language model of the natural language processing, including use of the SMILES notation for input, to adopt molecular fingerprints. Molecular fingerprints are the vectorized representations of molecules capturing precise details of atomic configurations and can be derived from molecular graphs, enabling calculations based on global molecular descriptors, feature position aware encoding of individual atom and bond features, and preserve the chemical identity of functional groups. They can be used to numerically describe properties of interest such as molecular structure, chemical properties such as molecular weight, hydrogen bond donor count, hydrogen bond acceptor count, topological polar surface area, formal charge, complexity, color/form, odor, taste, boiling point, melting point, stability/shelf life, reactivity etc.

The system can use the vectorization process prior to the training process, allowing the system to simultaneously consider all the molecular fingerprints (e.g., descriptors) during the training process and speeding up the overall learning. The molecules presented to the system can then retain their molecular structure, allowing the system to provide analysis and determinations in both a forward and a backward direction. The prediction provided by the system has advantages in that the geometry and characteristics of other molecules (or adjuvants) can be predicted. The system can use the deep learning architecture and find non-linear relationships in relatively large datasets. The use of vectors also enables building feature maps and rendering a score that indicates the degree of molecular overlap between the selected structures. The score can also be used to discover new synergistic combinations based on geometry.

### EXAMPLE 3

In one embodiment, the system can include a deep learning model that includes the naturel language processing model using the SMILES format. The system is created using a first portion of a dataset for training with the second portion used to test the system's accuracy and a third portion can used or reserved for fine-tuning the system predictive ability. The example, the first portion can be in the range of 50% to 80% of the dataset, the second portion can be in the range of 20% to 50% and the third portion in the range of 20% to 50%.

In one embodiment, molecular fingerprints, which can be vectorized representations of molecules capturing precise details of atomic configurations, can be used. Molecular fingerprints can be derived from molecular graphs, enabling calculations based on global molecular descriptors, feature position-aware encoding of individual atom and bond features, and preserve the chemical identity of functional groups. This can provide the advantage that these vectors (e.g., descriptors) can be used to numerically describe properties of interest such as molecular structure, chemical properties such as Molecular Weight, Hydrogen Bond Donor Count, Hydrogen Bond Acceptor Count, Topological Polar Surface Area, Formal Charge, Complexity, Color/Form, Odor, Taste, Boiling Point, Melting Point, Stability/Shelf Life, etc. and reactivity. These descriptors can be included in the database. Molecular fingerprints can also provide advantages that include including allowing the simultaneously consideration of all descriptors during the training process and speeding up the overall learning; the retention of their molecular structure, allowing calculations in both forward and backward directions and can be useful when making predictions, as the geometry and characteristics of other molecules (or adjuvants) can be predicted and then searched for using similarity approaches. This approach, supported by the selected deep learning architecture, allows finding non-linear relationships in relatively large datasets.

The neural network would initially only use a portion of the existing data as a training dataset with the remaining portion used as a testing database to verify the accuracy. A third portion can be used for fine-tuning the hyperparameter of the computerized method and can be referred to as an evaluation dataset. This method is advantageous to prevent overfitting. The testing portion may not be used to train the model. The use of vectors also enables building feature maps, rendering a score, such as in the last layer, that can indicate the degree of molecular overlap between the selected structures. The score can also be used to discover new synergistic combinations based on geometry.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A computerized method of predicting the formation of deep eutectic solvents, from a mixture of molecules comprising:
providing an initial dataset of existing deep eutectic solvents, divided into a training portion and a testing portion;
providing an initial group of molecules,
providing a set of representations for the initial group of molecules,
generating a set of mixtures,
predicting their probability of formation using a set of artificial neural network algorithms and the initial group of molecules,
selecting a first mixture from the set of mixtures predicted by the set of artificial neural network algorithms, wherein the first mixture comprises molecules not present in the training portion,
comparing test mixture within the set of mixtures with the testing portion to provide a confidence score for each artificial neural network algorithm in the set of artificial neural network algorithms, and,
providing the set of artificial neural network algorithms in confidence score order to a user.

2. The method of claim 1 including providing the initial dataset having a set of compounds using a simplified molecular input line entry system.

3. The method of claim 1 including providing the initial group of molecules using a simplified molecular input line entry system.

4. The method of claim 1 including providing the initial group of molecules using a vectorized representation of the initial group of molecules.

5. The method of claim 1 including providing the initial group of molecules using a vectorized representation of the initial group of molecules.

6. The method of claim 1 including providing the initial dataset wherein each deep eutectic solvent has a designation of stable or not stable.

7. The method of claim 6 including generating a predicted mixture using a first artificial neural network algorithm, comparing a probability of formation of the generated set of mixtures with the testing portion includes, comparing a first stability value of the test mixture with a second stability value of the first mixture.

8. The method of claim 1 wherein the training portion has a set of records numbering greater than 50% of the records in the initial dataset.

9. The method of claim 1 wherein the training portion has a set of records in a range of 50% to 90% of the records in the initial dataset.

10. A computerized method of predicting deep eutectic mixtures from a molecule comprising:
providing an initial dataset of existing deep eutectic solvents having a training portion and a testing portion;
providing an initial molecule,
generating a set of predictive deep eutectic solvents according to an artificial neural network and the initial molecule,
selecting a test deep eutectic solvent from the set of deep eutectic solvents wherein the test deep eutectic solvents is not present in the training portion,
comparing test deep eutectic solvents with the testing portion to provide a confidence score, and,
providing the confidence score to a user.

11. The method of claim 10 wherein the training portion has a set of records numbering greater than 50% of the records in the initial dataset.

12. The method of claim 10 wherein the training portion has a set of records in a range of 50% to 90% of a number records in the initial dataset.

13. The method of claim 10 wherein generating a set of predictive deep eutectic solvents includes generating a stability probability according to the artificial neural network and the training portion.

14. The method of claim 13 including displaying a subset of the set of predictive deep eutectic solvents having a stability probability higher than 50%.

15. The method of claim 10 wherein:
the set of predictive natural deep eutectic solvents is a first set of predictive deep eutectic solvents;
providing a desired molecule;
generating a second set of predictive deep eutectic solvents according to an artificial neural network and the desired molecule;
providing an accurate score to the set of predictive deep eutectic solvents; and,
providing the accurate score to the artificial neural network to recursively train the artificial neural network.

16. The method of claim 10 wherein the artificial neural network includes a binary classifier.

17. The method of claim 10 wherein the training portion includes randomly generated deep eutectic solvents.

18. A computerized method of predicting deep eutectic mixtures from a molecule comprising:
providing an artificial neural network trained with a dataset of existing deep eutectic solvents having a training portion and a testing portion wherein the training portion has a record size larger than that of the testing portion;
providing an initial molecule,
generating a set of predicted deep eutectic solvents according to an artificial neural network and the initial molecule,
generating a set of predicted deep eutectic solvents and,
displaying the set of predicted deep eutectic solvents to a user.

19. The method of claim 18 including generating a confidence value for each of the predicted deep eutectic solvents in the set of predicted deep eutectic solvents.

20. The method of claim 19 including displaying a subset from the set of predicted natural deep eutectic solvents having a confidence value greater than a predetermined value.
